(19) United States
(12) Patent Application Publication
(10) Pub. No.: US 2006/0020308 A1
(43) Pub. Date: Jan. 26, 2006

(54) Light therapy device heat management

(76) Inventor: James Scott Muldner, Reno, NV (US)

Correspondence Address: Scott Muldner, 4750 Turbo Circle, Reno, NV, 89502, US

(21) Appl. No.: 10/894,623

(22) Filed: Jul. 20, 2004

(51) Int. Cl. A61N 5/06

(52) U.S. Cl. 607/88; 607/90

(57) ABSTRACT

This device incorporates an array of light emitting diodes (LED) of several specific wavelengths that are used to provide therapy to visible and invisible living tissue or skin disorders that react to various wavelengths of light. Furthermore the device uses a combination of high level of perforation metal core circuit boards, heat transfer pads, heat sinks and forced air cooling and electronic thermal management to achieve continuous high intensity light output to therapy areas along with long LED life. The invention is using replaceable LED modules enabling device maintenance by user operators not trained as maintenance technicians.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

1. Field of Invention

This invention relates generally to the field of medical living tissue using light therapy in and more specifically to improvement of light devices used in therapy of visible and invisible skin disorders that react to various wavelengths of light.

2. Prior Art

Diamantopolus¹, et al. in U.S. Pat. No. 4,930,504 (Jun. 5, 1990) and other references teach us that certain wavelengths of light ranging from 600 nm thru 1500 nm are effective in biostimulation of tissue. For many years, high-powered highly focused lasers have been used to cut and destroy tissues in many surgical techniques. More recently, low powered lasers, less sharply focused, which do not cut or destroy tissues have been found to, or thought to, affect numerous metabolic processes, including cell division, cyclic-AMP metabolism, oxidative phosphoration, hemoglobin, collages and other protein synthesis, leukocyte activity, tumor growth, production of macrophage cells, and wound healing. Read, for example, Harry T. Whelan² et al. “Medical Applications of Space Light-Emitting Diode Technology-Space Station and Beyond”, 15 pages, CP458 Space Technology and Applications International Forum-1999; Harry T. Whelan³ et al. Effect of NASA Light-Emitting Diode Irradiation on Wound Healing. Journal of Clinical Laser Medicine and Surgery, vol 19, Nov. 6, 2001; Mary Ann Liebert⁴, Inc pp 305-314. “LEDs Illuminate the Future of Light Based Skin Rejuvenation” American Society for Dermatological Surgery, Apr. 10, 2003—Schamburg, Ill.

McDaniel⁵, in U.S. Pat. No. 6,663,659 (Dec. 16, 2003), discusses LED light therapy technology without addressing the servicing and heat management of the present invention. Pecukonis⁶, in U.S. Pat. No. 6,471,716 (Jun. 11, 2002), addresses living tissue therapy in the infrared light spectrum and does not address problems concerning LED array replacement or heat control problems without light pulsing

There is a need for LED arrays to be able to concentrate more light in a given area to reduce tissue treatment times, concentrate the focus of light on controlled areas, reduce the cost and weight of the devise, improve LED life, and reduce the maintenance cost and down time when an LED burns out or other light source malfunctions. The limitation to increasing light output for a given area has been the overheating of the LED and other components causing short component life. Further heat management avoids difficult to handle and often unacceptable current fluctuations in LED arrays, Ito,⁷ et al. in U.S. Pat. No. 4,720,480 (Jan. 19, 1988), suggests materials are available that enable rapid heat transfer of heat from one surface to another with very little heat retained from the transfer. Lev⁸, in U.S. Pat. No. 6,301,107 (Oct. 9, 20001), states thermosyphoning pipes incorporated into computer heat generating devices can be effective in enhancing heat removal.

Hsu⁹, in U.S. Pat. No. 6,705,393 (Mar. 16, 2004), shows ceramic heat sinks having a micro-pore structure can greatly enhance heat dissipation. Bolognia¹⁰, et al in U.S. Pat. No. 6,373,696 (Apr. 2, 2002), says thermal transfer pads improve the efficiency of heat transfer. Hochstein¹¹ et al, in U.S. Pat. No. 6,582,100 (Jun. 24, 2003), explains very well the advantages and limitations of various methods of assembling LED mounting systems. Also, there is a need for the device operator to be able to select various wavelengths of light for the patient without having to move the device or change light arrays. In addition, there is a need for the device operator, not trained as an maintenance technician, to be able to replace a failed LED array.

### BRIEF SUMMARY OF THE INVENTION

The primary object of the invention is achieving higher light energy outputs per unit area with minimum LED life of 5000 hours by using highly perforated metal core circuit boards as an essential part of a heat management system.

Further objects of the invention include:

1. Higher light intensity, to a given area thus reducing treatment time, by using metal core circuit board heat management.

2. Improving light therapy devices by lowering operating costs by enabling user/operator maintenance.

3. Using “blue light” wavelength, optimized for acne vulgaris with over 2800 LED devices operating in the 400 to 500 nm range

4. Using “red light” wavelength which can be selected for wound healing and operating in the 600 to 880 nm range.

5. Using combinations of above which can be operator selected to optimize treatment times and effectiveness.

6. Extending LED life utilizing improved heat management.

7. Utilizing user replaceable plug-in LED modules to enable device repair by non-maintenance technicians.

8. Enabling lower maintenance costs utilizing improved heat management.

9. Simplifying design to enable improved economic benefits.

10. Improving dependability in technology of LED array modules by use of this device and it's associated heat management design.

11. Qualifying the invention so the FDA will accept the device as a LED light therapy device with nonsignificant risk.

Other objects and advantages of the present invention will become apparent from the following descriptions, taken in connection with the accompanying drawings, wherein, by way of illustration and example, an embodiment of the present invention is disclosed.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a perspective view of the light therapy device

FIG. 2 shows the operator/user removable modules

FIG. 3 is the exploded view of the LED module.

FIG. 4 is side section view of the completed module assembly.

FIG. 5 is the rear view of completed module assembly

FIG. 6 is the connector sockets that hold the modules in place

FIG. 7 is a block diagram of the power control.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure, or manner.

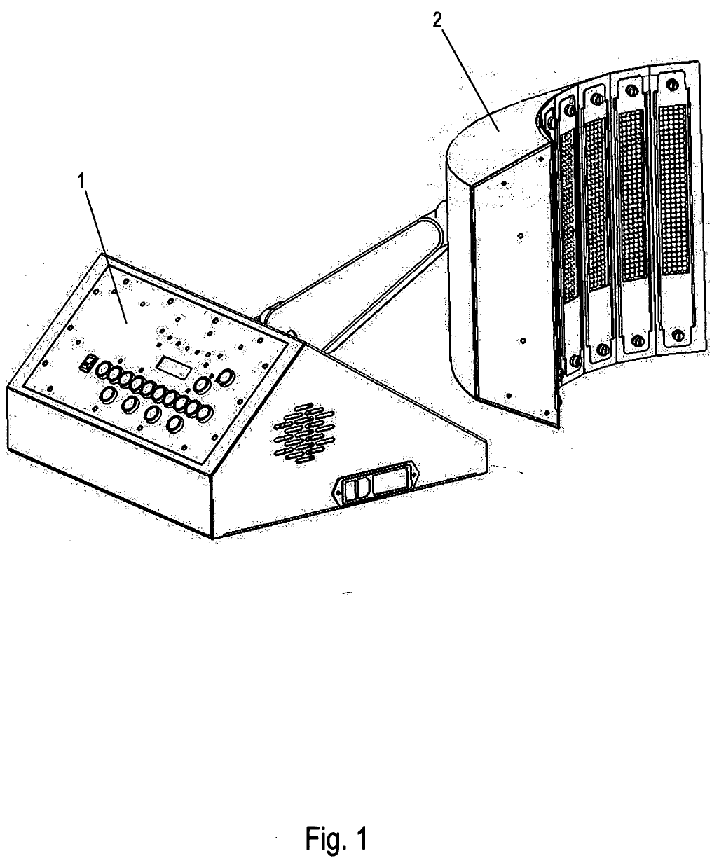

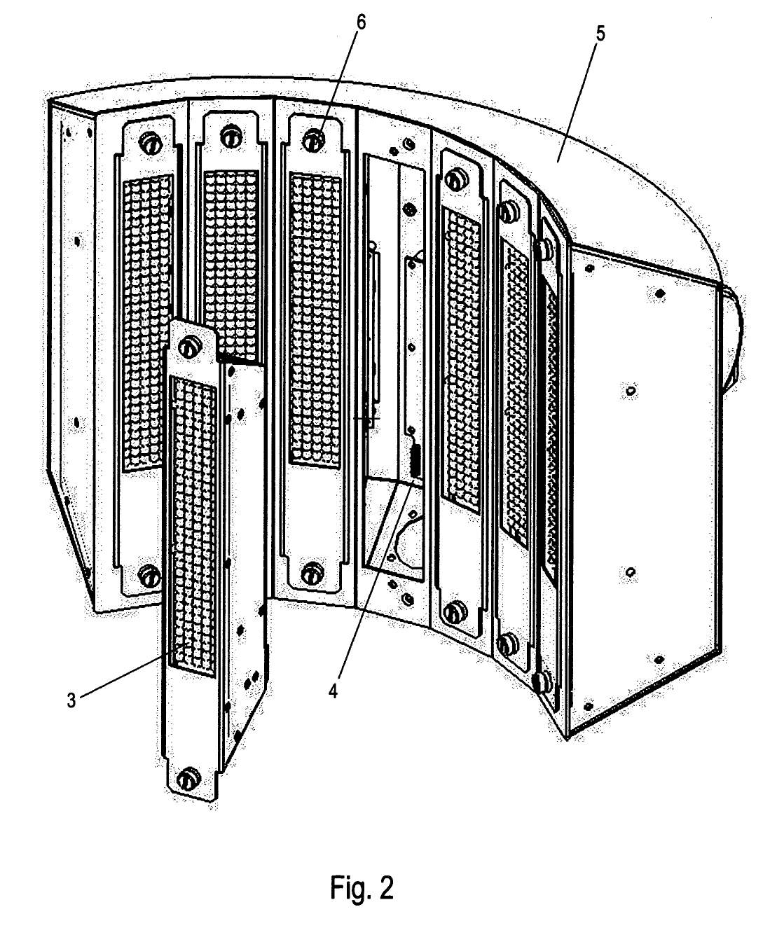

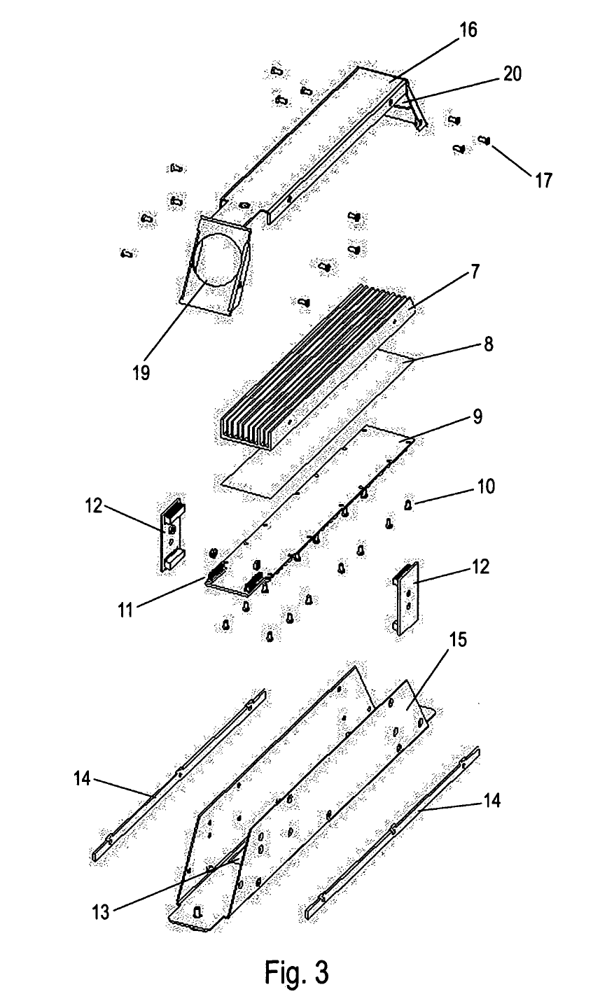

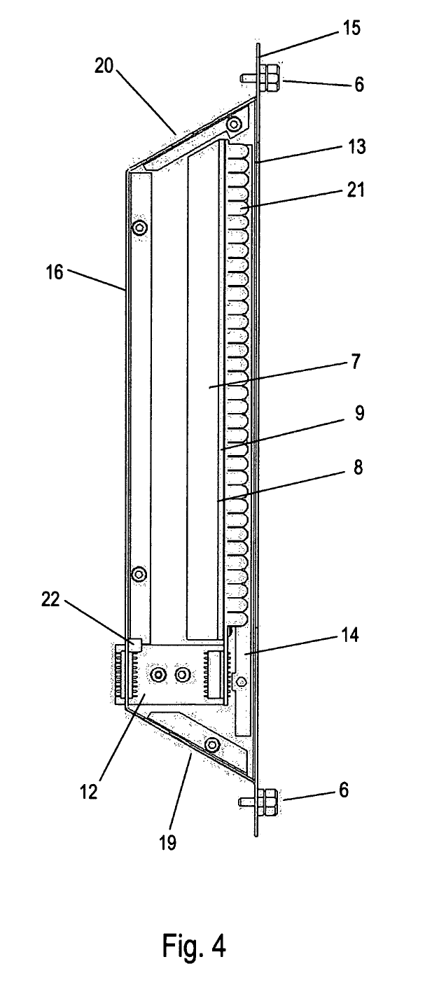

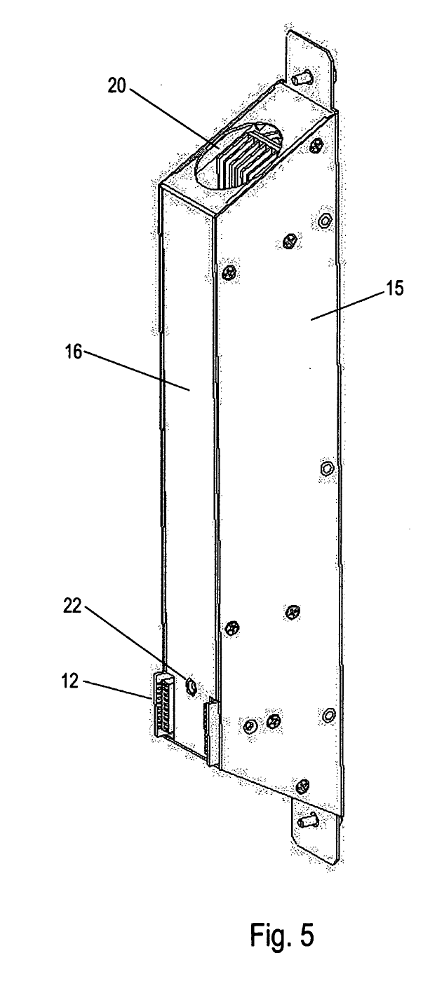

Turning to FIG. 1, one sees a perspective view of a version of the completed light therapy device including a operator control panel (1) and the light therapy device (2) incorporating the present invention. In accordance with an important feature of the present invention, FIG. 2 shows a version of the light therapy device containing the operator removable LED array module (3), and the power transfer connector (4) for attaching the LED array module (3) to the shroud (5). The mounting screws (6) are used to mount or dismount the LED array module (3) without the use of special tools to the shroud (5). Only a common screwdriver is required. To accomplish an important feature of the invention, there is shown in FIG. 3 an exploded view of the essential elements of the LED module (3) assembly. The module is constructed starting with the heatsink (7) to which attaches a thermal pad (8). Following is the metal core circuit board (9) containing the multiple LED (on the opposite side, not shown) which is assembled as a unit with mounting screws (10) to thermal pad (8) and heatsink (7). The left and and right modular extender boards (12) are plugged into the modular extender connectors (11). The lens (13) is mounted using right and left lens supports (14) onto front modular cover (15). The sub assembly unit consisting of heatsink (7) thermal pad (8) metal core circuit board (9) mounting screws (10) and modular extender boards (12) are mounted into front modular cover (15). The back modular cover (16) is mounted using mounting screws (17) onto the sub assembly consisting of parts (7).(8).(9).(10).(11).(12).(13),(14) and (15). An essential feature of this invention is the size and location of lower intake air vent (19) and upper exhaust air vent (20). This collection of components and configurations contained in FIG. 3 coupled with conventional air circulating fans are essential elements of the invention enabling high density electronic component thermal management enabling extended operating time without the need for auxiliary cooling systems using water or other wet fluids. FIG. 4 is the section view of the completed LED module assembly with a side of the front modular cover not shown. This view shows essential features of the invention not shown clearly in previous FIG. 1,2, or 3. Shown clearly are the lens (13), the LEDs (21), the LED module to shroud guide pin assembly (22), and the air path from the air intake vent (19) flowing upward between the LED (21) and lens (13) and across the heat sink (7) with the air exiting through exit air vent (20). The operator mounting screws (6) are for enabling the operator to secure the LED array module (3) to the shroud (5) after guiding the module extender boards (12) onto power transfer connector (4) using guide pin assembly (22). FIG. 5 shows the rear of the module showing clearly the modular extender boards (12) and the guide pin assembly (22) as well as the exit air vent area (20).

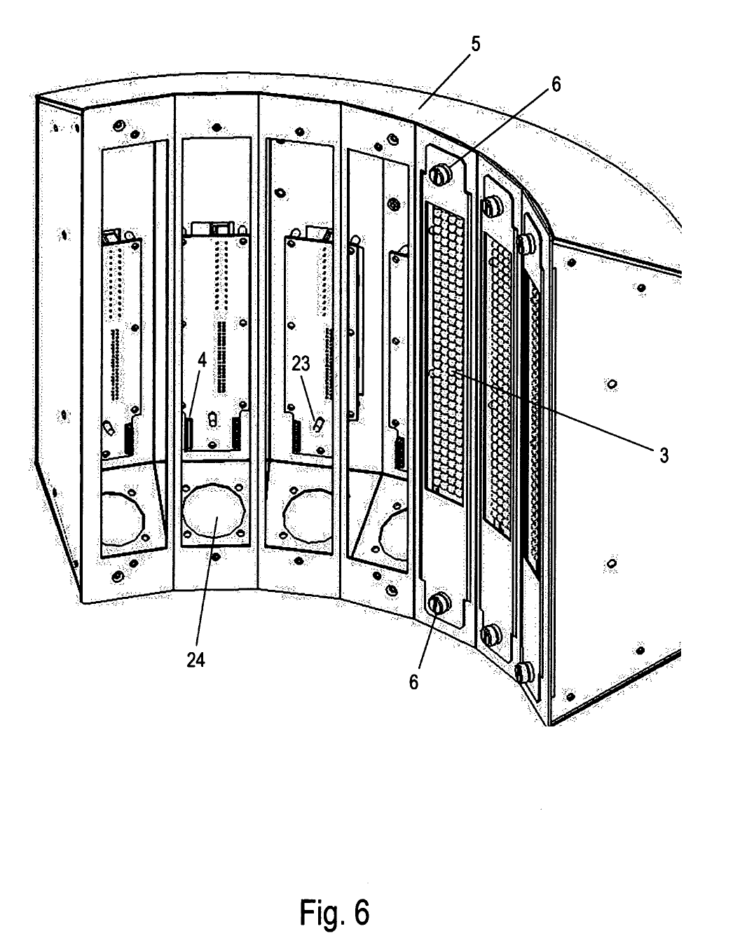

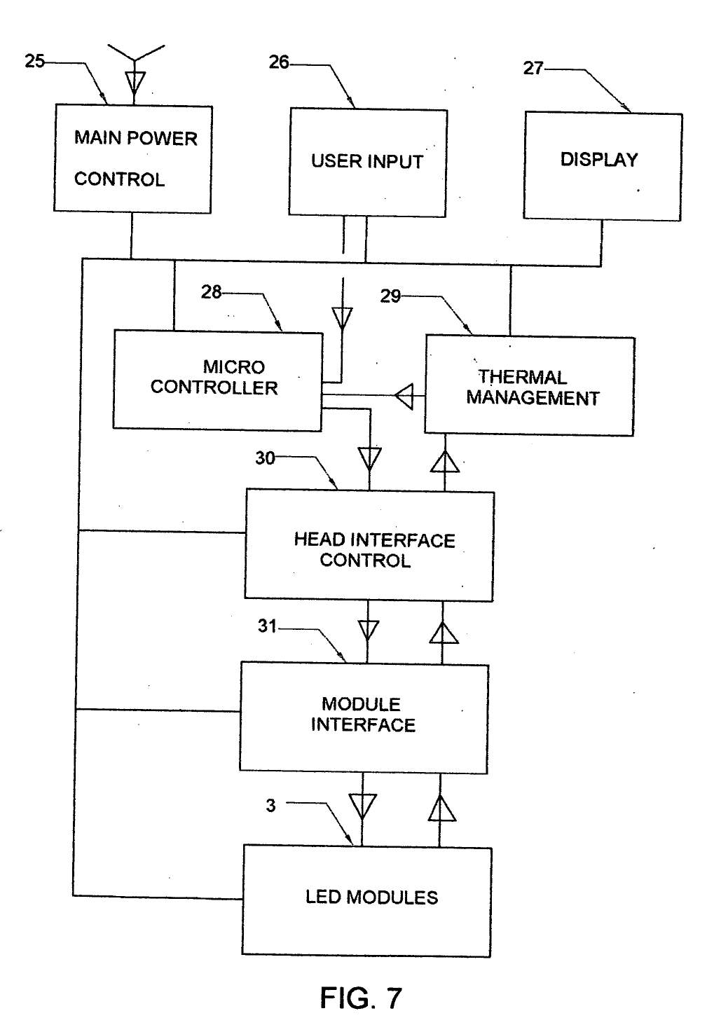

Turning to FIG. 6 is a view showing the connecting method holding the LED array module (3) to the shroud (5). by aligning the guide pin assembly (22) onto the guide pin (23) enabling the connecting of modular extender board (12) with power transfer connector (4) Also shown in FIG. 6 is the intake air fan mounting area (24) FIG. 7 shows a block diagram of the controls which are an essential component of the invention. Electric power enters the device at (25) where it is transformed into appropriate voltages, currents and polarity to energize user input (26) display (27) microcontroller (28) thermal management (29) head interface control (30) module interfaces (31 and LED modules (3). User input block (26) outputs signals to the micro controller (27) where appropriate signals are sent to the head interface (30) and to initiate thermal management (29). Signals passing from the head interface (30) are then distributed to the module interfaces (31) onto the LED modules (3) where the selected wavelengths of light are mixed, fixed or pulsed for the selected power levels and time intervals for the desired light therapy treatment. Thermal conditions for all of the heat generating components are managed by input into the thermal management component (29) from which appropriate electronic signals are sent to the micro controller (28) for management of heat conditions of each of the heat generating components.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

1. A light therapy device, having replaceable multiple LED array modules used in therapy of visible and invisible skin or tissue disorders that react to various wavelengths of light and providing increased light output per unit area, thus reducing therapy time.

2. The device in claim 1 uses high level of perforation metal core circuit boards in cooperation with heat transfer pads and heat sinks and surface-flow air movement to achieve continuous high output heat dissipation with light outputs of minimum 100 mw/sqcm

3. The device in claim 1 uses multiple LED power levels of mixed wavelengths, fixed wavelengths or pulsed wavelengths.

4. The device in claim 1 utilizing LED array modules that can be replaced on site by non-technician trained user/operators.
